(19) United States
(12) Patent Application Publication
(10) Pub. No.: US 2003/0040672 A1
(43) Pub. Date: Feb. 27, 2003

(54) Surgical operation apparatus

(75) Inventors: Gen Ogura, Tokyo (JP); Hidenori Kato, Tokyo (JP); Shigeo Kimura, Tokyo (JP)

Correspondence Address: MARGER JOHNSON & MCCOLLOM PC, 1030 SW MORRISON STREET, PORTLAND, OR, 97205, US

(73) Assignee: Aloka Co., Ltd., Tokyo (JP)

(21) Appl. No.: 10/206,842

(22) Filed: Jul. 26, 2002

(30) Foreign Application Priority Data
Jul. 26, 2001 (JP) 2001-225432

(51) Int. Cl. A61B 8/00

(52) U.S. Cl. 600/437

(57) ABSTRACT

Disclosed herein is a surgical operation apparatus which comprises a tip member for outputting ultrasound vibration and/or high-frequency coagulation current, a handpiece provided with the tip member, a circuit for supplying the high-frequency coagulation current to the tip member and a circuit for supplying the ultrasound vibration to the tip member. The surgical operation apparatus includes an ultrasound vibration mode for mainly fragmenting tissue in a surgical operation region by outputting the ultrasound vibration from the tip member, and a coagulation mode for mainly performing hemostasis of the tissue by outputting the high-frequency coagulation current from the tip member. The ultrasound vibration mode and the coagulation mode are switchable by a control device, wherein during the coagulating mode, a predetermined coagulation mode ultrasound vibration is also supplied to the tip member, thereby suppressing the tissue from being burnt to be stuck to the tip member.

### BACKGROUND OF THE INVENTION

1. Field of the Invention

The present invention relates to a surgical operation apparatus having functions of fragmenting biological tissue using ultrasound vibration and incising and/or coagulating the tissue using high-frequency current.

2. Description of the Related Art

Conventionally, there have been used surgical operation apparatuses such as an electric knife and an ultrasound surgical apparatus (ultrasound aspirator) in surgical operations.

The electric knife is a surgical apparatus which performs incision (cutting) and hemostasis while cauterizing biological tissue by electric power concentration that occurs at a tip of a knife-shaped electrode when high-frequency current flows to a patient placed between the knife-shaped electrode and a counter electrode. It has an advantage in that a surgical operation can be performed with less amount of bleeding.

Among electric knives that have been used in recent years, there is an electric knife that can selectively use high-frequency cutting current mainly used in incising tissue and high-frequency coagulation current mainly used in coagulating tissue. In general, in such an apparatus, high-frequency current whose wave form becomes substantially a pure sine wave is used as the high-frequency cutting current and high-frequency current whose waveform becomes a damped sine wave is used as the high-frequency coagulation current.

On the other hand, the ultrasound surgical apparatus is a surgical apparatus which fragments biological tissue by ultrasound vibration supplied to a tip member (or horn) provided at a tip end of a handpiece. It has an advantage in that tissue can be selectively fragmented, in particular, it is advantageous when tissue around a blood vessel which has high elasticity is fragmented with leaving the blood vessel.

Such ultrasound surgical apparatuses have a function of not only fragmenting tissue at a surgical operation region by ultrasound vibration but also aspirating blood and fragmented tissues and the like in the surgical operation region. Such an ultrasound surgical apparatus fragments tissue in a surgical operation region by ultrasound vibration which occurs at a tip member with aspirating blood and fragmented tissues from an aspiration opening provided in the tip member.

In addition, in recent years, there is also known a surgical operation apparatus having a tip member to which both high-frequency current and ultrasound vibration can be selectively supplied, thereby possessing both the functions of the electric knife and the ultrasound surgical apparatus.

However, in the conventional surgical operation apparatus of this type, switching operations of ON/OFF of an output of high-frequency current and an output of ultra sound vibration should be performed separately. Thus, an assistant for the manipulation of the apparatus is required in addition to a doctor who performs treatment with the surgical operation apparatus. Therefore, the manipulation is complicated, and there is a case that erroneous manipulations occur.

In addition, in the conventional surgical operation apparatus, there is no cooperative association between an output of the ultrasound vibration at the time of an output of the high-frequency current and functions added to the ultrasound aspirator such as an irrigation liquid supply function and an aspiration function. As a result, it is difficult to realize a function that is most appropriate for each surgical operation and the technique required for the operation.

### SUMMARY OF THE INVENTION

The present invention has been made in view of the above problem, and it is an object of the present invention to provide a surgical operation apparatus that can be conveniently and safely operated.

In order to achieve the above object, the present invention is directed to a surgical operation apparatus which comprises a tip member for outputting ultrasound vibration and/or high-frequency coagulation current; a handpiece provided with the tip member; means for supplying at least the high-frequency coagulation current to the tip member; means for supplying the ultrasound vibration to the tip member, and control means for controlling the ultrasound vibration supplying means and the high-frequency coagulation current supplying means. In this apparatus, the supply of the high-frequency coagulation current to the tip member and the supply of the ultrasound vibration to the tip member are performed in a cooperatively associated manner as needed.

In the present invention, it is preferred that the apparatus includes an ultrasound vibration mode for mainly fragmenting tissue in a surgical operation region by outputting the ultrasound vibration from the tip member, and a coagulation mode for mainly performing hemostasis of the tissue by outputting the high-frequency coagulation current from the tip member, wherein the ultrasound vibration mode and the coagulation mode being switchable by the control means, wherein during the coagulating mode, the ultrasound vibration supplying means supplies a predetermined coagulation mode ultrasound vibration to the tip member.

This makes it possible to suppress the tissue from being burnt to be stuck to the tip member, thereby enabling to use the surgical apparatus for a long time without adhesion of the tissue to the tip member.

In this case, it is preferred that the predetermined coagulation mode ultrasound vibration is set to a level that can suppress the tissue from being burnt to be stuck to the tip member.

Preferably, the predetermined coagulation mode ultrasound vibration is set to a level that is approximate to 30 to 70% of the maximum vibration output of the ultrasound vibration mode. Also, preferably, a vibration speed of the tip of the tip member is in the range of 220 [cm/s] to 2200 [cm/s] when the coagulation mode ultrasound vibration is supplied to the tip member.

In the surgical operation apparatus of the present invention, it is also preferred that the apparatus further comprises means for aspirating liquid or fragmented tissue in the vicinity of the surgical operation region from the tip of the tip member, the aspirating means is adapted to stop during the coagulation mode.

Moreover, it is also preferred that the apparatus further comprises means for supplying irrigation liquid to the surgical operation region from the vicinity of the tip of the tip member, and the irrigation liquid supplying means is adapted to supply irrigation liquid in both the ultrasound vibration mode and the coagulation mode.

In this case, it is preferred that the amount of the irrigation liquid to be supplied to the surgical operation region can be set separately in each of the ultrasound vibration mode and the coagulation mode.

Furthermore, in the surgical operation apparatus of the present invention, it is preferred that the control means includes a hand switch provided on the handpiece to be operated by a hand of an operator. Further, it is also preferred that the control means includes a foot switch to be operated by a foot of an operator.

The above and other objects, structures and advantages of the present invention will be more apparent when the following detailed description of the embodiments is considered in conjunction with the accompanied drawings.

### BRIEF DESCRIPTION OF THE DRAWINGS

FIG. 1 is a block diagram which shows an over all structure of a surgical operation apparatus according to the present invention.

FIG. 2 is a cross-sectional view of a handpiece used in the surgical operation apparatus.

FIG. 3 is a timing chart of operations of a high-frequency current generating device, an ultrasound vibration current generating device, an aspiration device and a irrigation liquid supply device in an cutting mode of the surgical operation apparatus of the present invention.

FIG. 4 is a timing chart of operations of the high-frequency current generating device, the ultrasound vibration current generating device, the aspiration device and the irrigation liquid supply device in a coagulation mode of the surgical operation apparatus of the present invention.

FIG. 5 is a timing chart of operations of the high-frequency current generating device, the ultrasound vibration current generating device, the aspiration device and the irrigation liquid supply device in an ultrasound vibration mode of the surgical operation apparatus of the present invention.

FIG. 6 is a front view which illustrates an adjustment panel provided in connection with a control device (controller) of the surgical operation apparatus of the present invention.

### DETAILED DESCRIPTION OF THE PREFERRED EMBODIMENTS

Hereinbelow, preferred embodiments of a surgical operation apparatus according to the present invention will be described based on the accompanying drawings.

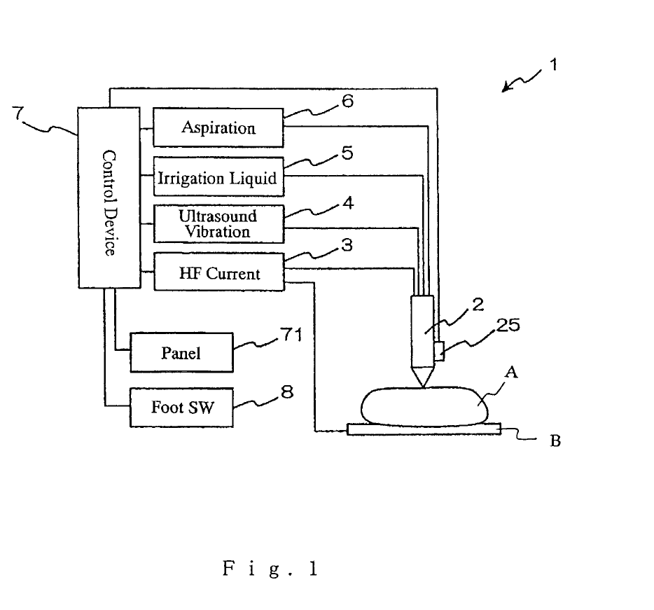

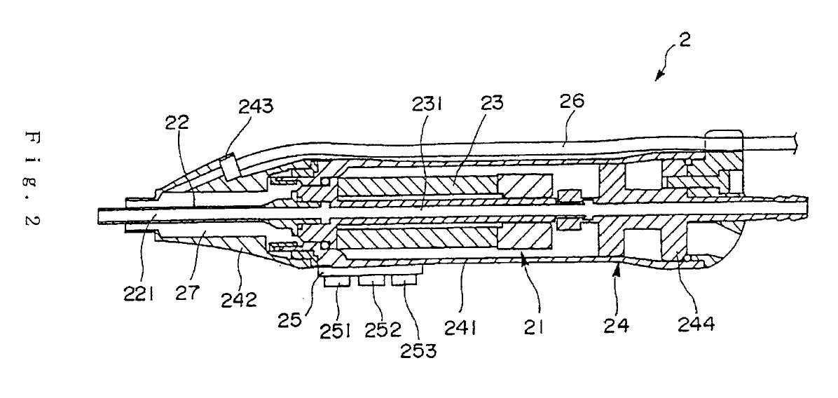

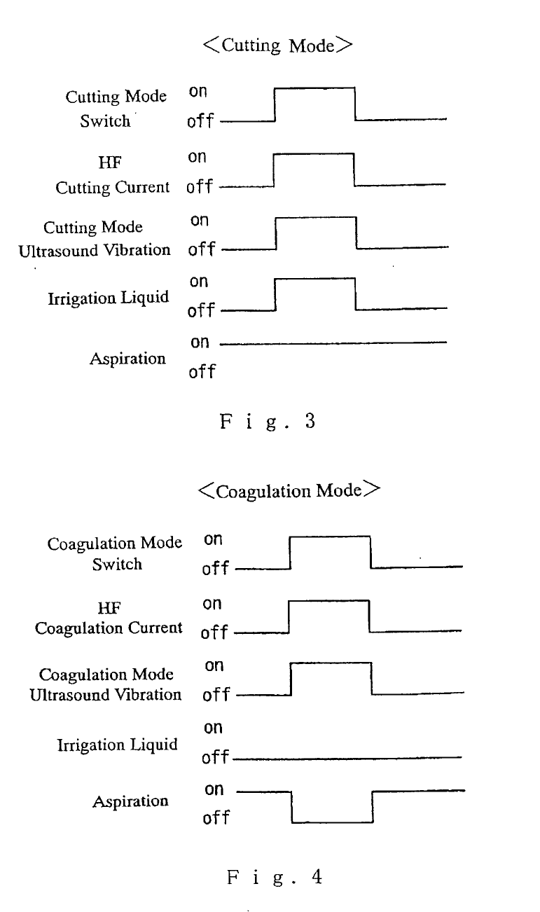

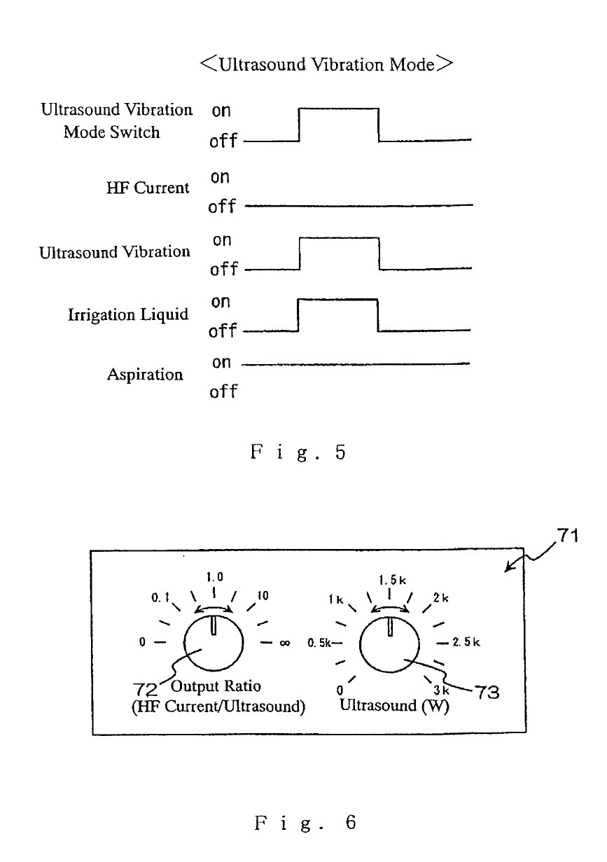

In the accompanying drawings, FIG. 1 is a block diagram which shows an overall structure of a surgical operation apparatus according to the present invention; FIG. 2 is a cross-sectional view of a handpiece used in the surgical operation apparatus; FIG. 3 is a timing chart of operations of a high-frequency current generating device, an ultrasound vibration current generating device, an aspiration device and a irrigation liquid supply device in an cutting mode of the surgical operation apparatus of the present invention; FIG. 4 is a timing chart of operations of the high-frequency current generating device, the ultrasound vibration current generating device, the aspiration device and the irrigation liquid supply device in a coagulation mode of the surgical operation apparatus of the present invention; FIG. 5 is a timing chart of operations of the high-frequency current generating device, the ultrasound vibration current generating device, the aspiration device and the irrigation liquid supply device in an ultrasound vibration mode of the surgical operation apparatus of the present invention; and FIG. 6 is a front view which illustrates an adjustment panel provided in connection with a control device (controller) of the surgical operation apparatus of the present invention.

In this regard, it is to be noted that in the following description, the left side in FIG. 2 is referred to as “tip side” or “tip” and the right side in FIG. 2 is referred to as “rear side” or “rear” for simplifying the description.

As shown in FIG. 1, a surgical operation apparatus of the present invention includes a handpiece 2; a high-frequency current generating device (high-frequency current supplying means) 3 for supplying high-frequency current to the handpiece 2; an ultrasonic vibration current generating device (ultrasonic vibration supplying means) 4 for supplying current for generating ultrasonic vibration to the handpiece 2; a irrigation liquid supply device 5 for supplying irrigation liquid to the vicinity of a surgical operation region; an aspiration device 6 as aspirating means for aspirating liquid, fragmented tissue and the like in the vicinity of a surgical operation region; a control device 7 as controlling means for controlling each of these devices. The surgical operation apparatus is further provided with a hand switch 25, an adjustment panel 71 and a foot switch 8 that are connected to the control device 7. Note that reference symbol A in the figure indicates a patient for which this surgical operation apparatus is used and reference symbol B indicates a counter electrode disposed below the patient.

The structure of each element will be hereinafter described in detail.

As shown in FIG. 2, the handpiece 2 is provided with a surgical operation unit 21 and a casing 24 for housing the surgical operation unit 21.

The surgical operation unit 21 is generally composed from a tip member 22 and an ultrasound vibrator 23 that is coupled to the tip member 22 for supplying ultrasound vibration to the tip member 22.

The tip member 22 is made of a material having resistance against ultrasound vibration such as titanium alloy or stainless steel, and it has both a function as a knife tip of an electric knife, that is, as an electrode, and a function as a tip of an ultrasound surgical apparatus, that is, as an ultrasound horn. In addition, this tip member 2 is connected to the high-frequency current generating device 3 by a conductor (not shown in FIG. 2). Further, this tip member 22 is formed into a hollow pipe having an axially extended aspiration passage 221 which aspirates there through liquid such as blood in the vicinity of a surgical operation region, fragmented tissue and the like.

The aspiration passage 221 extends from the tip of the tip member 22 toward the rear end thereof, and it is connected to the aspiration device 6 shown in FIG. 1 via a flow path 231 described later.

The ultrasound vibrator 23 is, for example, a bolt-fastening ultrasound vibrator, and it is constructed from a plurality of piezoelectric elements, electrodes and metal blocks placed on both sides of these elements and electrodes, and the like.

The tip member 22 is fixed to the tip portion of the ultrasound vibrator 23, and vibration of the ultrasound vibrator 23 is amplified to be transmitted to the tip member 22. In addition, provided on a central axis of the ultrasound vibrator 23 is the flow path 231 for delivering the liquid, the fragmented tissue and the like aspirated from the aspiration passage 221 of the tip member 22 to the aspiration device 6. In addition, this ultrasound vibrator 23 is connected to the ultrasound vibration current generating device 4 by a conductor (not shown).

As shown in FIG. 2, the casing 24 is constructed mainly from a casing main body 241, and a tip side cap 242 and a rear end side cap 244 which are located at the tip side and the rear end side of the casing main body 241, respectively.

The casing main body 241 is formed of a resin material substantially in a circular shape, and the surgical operation unit 21 is housed therein. In addition, the hand switch 25 is provided in this casing main body 241.

As shown in FIG. 2, the hand switch 25 is provided in a side portion on the tip side of the casing main body 241.

In addition, the hand switch 251 is provided with an cutting mode switch 251, a coagulation mode switch 252 and an ultrasound output mode switch 253. When the cutting mode switch 251 is turned on, an cutting mode, that is, a mode for mainly performing incising of biological tissue using high-frequency cutting (incising) current and cutting mode ultrasound vibration is selected. When the coagulation mode switch 252 is turned on, a coagulation mode, that is, a mode for mainly performing coagulation and hemostasis of blood or biological tissue using high-frequency coagulation current and coagulation mode ultrasound vibration is selected. In addition, when the ultrasound output mode switch 253 is turned on, an ultrasound vibration mode for fragmenting tissue and aspirating fragmented tissue only by ultrasound vibration is selected. Further, any one of the cutting mode switch 251, the coagulation mode switch 252 and the ultrasound output mode switch 253 is selected. Each of these switches is connected to the control device 7 by a conductor (not shown in FIG. 2).

Moreover, the foot switch 8 (see FIG. 1) connected to the control device 7 is also provided with three types of switches that have functions equivalent to those of the cutting mode switch 251, the coagulation mode switch 252 and the ultrasound vibration mode switch 253 provided in the hand switch 25.

When the switches of the identical mode of the respective foot switch 8 and hand switch 25 are simultaneously pushed, for example, when the cutting mode switch provided in the foot switch 8 and the cutting mode switch 251 provided in the hand switch 25 are simultaneously pushed, the manipulation of both the switches become valid.

On the other hand, when the switches of the mutually different modes of the respective foot switch 8 and hand switch 25 are simultaneously pushed, for example, when the cutting mode switch 251 of the hand switch 25 and the coagulation mode switch of the foot switch 8 are simultaneously pushed, the manipulation of the hand switch 25 becomes valid and the manipulation of the foot switch 8 becomes invalid. However, the present invention is not limited to this, and the manipulation of the hand switch 25 may be invalid and the manipulation of the foot switch 8 may be valid.

In addition, the casing main body 241 is provided with a irrigation liquid supply pipe 26 that is disposed along the external circumference surface of the casing main body 241 and supplies irrigation liquid to the tip portion of the tip member 22. The rear end side of the irrigation liquid supply pipe 26 is connected to the irrigation liquid supply device 5 shown in FIG. 1.

The irrigation liquid supply device 5 is constructed such that a supply amount of irrigation liquid can be changed according to a type or condition of high-frequency current supplied to the tip member 22 by the control of the control means 7.

As shown in FIG. 2, the tip side cap 242 is provided so as to cover the external circumference of the tip member 22. The tip side cap 242 is provided with a connection potion 243 to which the tip portion of the irrigation liquid supply pipe 26 is connected. In addition, the irrigation liquid supply path 27 is formed between the tip side cap 242 and the tip member 22. Irrigation liquid supplied from the irrigation liquid supply pipe 26 is supplied to the tip portion of the tip member 22 through the irrigation liquid supply path 27.

The high-frequency current generating device 3 is electrically connected to the tip member 22 of the hand piece 2 as described above, and it is constructed such that high-frequency cutting current and high-frequency coagulation current can be selectively supplied to the tip member 22. As described above, the high-frequency cutting current is high-frequency current with its waveform represented by substantially a pure sine wave and is preferably used in performing incising of tissue. In addition, the high-frequency coagulation current is high-frequency current with its waveform represented by a damped sine wave and is preferably used for coagulation, that is, hemostasis of tissue.

The ultrasound vibration current generating device 4 is connected to the ultrasound vibrator 23 in the handpiece 2, and it is constructed such that cutting mode ultrasound vibration used when the high-frequency cutting current is supplied from the high-frequency current generating device 3 (i.e., cutting mode), coagulation mode ultrasound vibration used when the high-frequency coagulation current is supplied from the high-frequency current generating device 3 (i.e., coagulation mode) and regular ultrasound vibration for the ultrasound vibration mode used when tissue is fragmented only by an ultrasound output (i.e., ultrasound mode) can be selectively supplied to the tip member 22. Here, the cutting mode ultrasound vibration, the coagulation mode ultrasound vibration and the ultrasound vibration for the ultrasound vibration mode maybe ultrasound vibrations of different three types having different amplitudes. In addition, a vibration speed of the tip of the tip member 22 is preferably 220 [cm/s] to 2200 [cm/s] when the cutting mode ultrasound vibration or the coagulation mode ultrasound vibration is supplied. In this regard, it is to be noted that the ultrasound vibration supplying means in the present invention includes the ultrasound vibration current generating device 4 and the ultrasound vibrator 23.

As described above, in the cutting mode and the coagulation mode, in particular, in the coagulation mode, when the coagulation mode ultrasound vibration is supplied to the tip member 22 in addition to the high-frequency coagulation current, an effect is realized in that tissue is prevented from being burnt to be stuck to the tip of the tip member 22, so that hemostasis effect does not decrease for a long time. In this case, the coagulation mode ultrasound vibration in the coagulation mode is set to a degree required for preventing tissue from being burnt to be stuck to the tip of the tip member 22. For example, the coagulation mode ultrasound vibration is set between approximately 30% to 70% of the maximum output of the regular ultrasound vibration in the ultrasound vibration mode, more preferably to approximately 50%.

The aspiration device 6 is connected to the aspiration passage 221 provided in the tip member 22. Liquid, fragmented tissue and the like in the vicinity of a surgical operation region are aspirated from the tip of the tip member 22 due to the aspiration by the aspiration device 6.

The control device 7 is usually provided with a microcomputer (CPU) and controls all of the high-frequency current generating device 3, ultrasound vibration current generating device 4, irrigation liquid supply device 5 and aspiration device 6. In this connection, it is to be noted that the controlling means in the present invention includes the control device 7, and the hand switch 25, the foot switch 8 and an adjustment panel 71 described later connected to the control device 7.

Hereinafter, the operation of the surgical operation apparatus of the present invention will be described.

When the cutting mode switch 251 of the hand switch 25 (or the foot switch 8) is turned on, the cutting mode is selected. In this mode, the high-frequency current generating device 3 and the ultrasound vibration generating device 4 output the high-frequency cutting current and the cutting mode ultrasound vibration, respectively, by the control of the control device 7 as shown in the time chart of FIG. 3. In addition, the irrigation liquid supply device 5 supplies irrigation liquid in synchronous with these outputs. Further, at this point, the aspiration by the aspiration device 6 is always performed regardless of whether or not the cutting mode switch 251 is turned on. In addition, an amount of the irrigation liquid in this case is set independently from the amount of the irrigation liquid used in the ultrasound vibration mode (described later). In this embodiment, an amount of irrigation liquid smaller than that in the ultrasound vibration mode is supplied to the tip portion of the tip member 22.

When the cutting mode switch 251 is turned off from this state, the cutting mode is released, and the output of the high-frequency cutting current and the output of the cutting mode ultrasound vibration and the supply of the irrigation liquid are stopped by the control of the control device 7 as shown in FIG. 3.

In addition, when the coagulation mode switch 252 of the hand switch 25 (or foot switch 8) is turned on, the coagulation mode is selected. In this mode, the high-frequency current generating device 3 and the ultrasound vibration generating device 4 output the high-frequency coagulation current and the current for the coagulation mode ultrasound vibration, respectively, by the control of the control device 7 as shown in the time chart of FIG. 4. In addition, the aspiration device 6 stops aspiration which has been being performed to this point in synchronous with these outputs. Further, when this coagulation mode is selected, the irrigation liquid supply device 5 always stops supply of the irrigation liquid regardless of whether or not the coagulation mode switch 252 is turned on.

When the coagulation mode switch 252 is turned off from this state, the coagulation mode is released, and the output of the high-frequency coagulation current and the output of the coagulation mode ultrasound vibration are stopped by the control of the control device 7 as shown in FIG. 4. In addition, the aspiration by the aspiration device 6 is re-started.

In addition, when the ultrasound output mode switch 253 of the hand switch 25 (or foot switch 8) is turned on, the ultrasound vibration mode is selected and the ultrasound vibration current generating device 4 outputs current for generating desired regular ultrasound vibration by the control of the control device 7 as shown in the time chart of FIG. 5. In addition, the irrigation liquid supply device 5 performs supply of irrigation liquid in synchronous with the output. Further, at this point, aspiration by the aspiration device 6 is always performed regardless of whether or not the ultrasound vibration mode switch 253 is turned on. In addition, a supply amount of irrigation liquid in this case is set independently from those in the cutting mode and the coagulation mode. In this embodiment, irrigation liquid more than that in the cutting mode or the coagulation mode is supplied to the tip portion of the tip member 22.

When the ultrasound vibration mode switch 253 is turned off from this state, the ultrasound vibration mode is released, and the output of the ultrasound vibration and the supply of irrigation liquid are stopped by the control of the control device 7 as shown in FIG. 5.

As described above, in this embodiment, according to each output of the high-frequency cutting current and the high-frequency coagulation current from the high-frequency current generating device 3, the cutting mode ultrasound vibration or the coagulation mode ultrasound vibration which is preferably set for the output is supplied. Thus, when the type of the high-frequency current is switched, it is unnecessary to perform a switching operation for the ultrasound vibration associated with the type of the frequency current, and switching of both the modes can be conveniently performed by operating either the hand switch 25 or the foot switch 8 once.

In addition, in this embodiment, when the coagulation mode, that is, a mode for coagulating blood and biological tissue using the high-frequency coagulation current is selected, the aspiration by the aspiration device 6 is stopped. Thus, the coagulation is performed without aspirating or removing coagulated tissue.

In addition, in this embodiment, when the coagulation mode is selected, the supply of irrigation liquid by the irrigation liquid supply device 5 is stopped.

Thus, when the high-frequency coagulation current is supplied, a coagulating function of the high-frequency coagulation current is not impaired due to decrease in an electric current density by the irrigation liquid.

Further, the present invention is not limited to the above-described embodiment, and it is possible to construct the surgical operation apparatus such that irrigation liquid of which flow rate is set separately from those of the cutting mode and the ultrasound vibration mode is supplied to the tip portion of the tip member 22. In this case, carbonization of a coagulated region can be suppressed by supplying the irrigation liquid of a degree that does not cause the coagulating function of the high-frequency coagulation current to fall significantly. In addition, adhesion of tissue to the tip portion of the tip member 22 can also be suppressed by supplying the irrigation liquid.

In addition, by performing aspiration during the coagulation mode, coagulation can be performed while removing the fragmented tissue.

In this embodiment, the surgical operation apparatus is constructed such that a ratio of an output of the high-frequency current and an output of the ultrasound vibration that are used when the cutting mode or the coagulation mode is selected can be adjusted by an adjustment panel 71 provided in the control device 7.

A configuration of the adjustment panel 71 will be hereinafter described.

As shown in FIG. 6, the adjustment panel 71 is provided with an output ratio adjustment knob 72 and an output level adjustment knob 73. Each of these knobs is formed in substantially a cylindrical shape, respectively. Each knob is operated by rotating it appropriately with an operator's hand.

The output ratio adjustment knob 72 has a function as “first adjustment means” for adjusting a ratio of a magnitude of an output of the high-frequency current and a magnitude of an output of the ultrasound vibration. The output ratio adjustment knob 72 also has another function as “holding means” for holding the output ratio adjusted by the output ratio adjustment knob 72.

Values of ratios of an output electric power from the high-frequency current generating device and an output electric power from the ultrasound vibration generating device, which are represented by the following expression (1), are calibrated around the output ratio adjustment knob 72.

Output ratio=Output of high frequency current [W]/Output of ultrasound vibration [W] (1)

Then, by rotating the output ratio adjustment knob 72, the output ratio control device which is interlocked with a rotation shaft of the output ratio adjustment knob 72 is operated, and thereby the ratio of the output of the high-frequency current and the output of the ultrasound vibration can be adjusted. In this embodiment, if the output ratio adjustment knob 72 is set to zero, only the ultrasound vibration is outputted, and if it is set to infinity, only the high-frequency current is outputted.

In addition, the output ratio adjustment knob 72 is constructed such that a position at the point when it stops rotation is maintained, whereby a value of the output ratio in that position is held.

On the other hand, the output adjustment knob 73 has a function as “second adjusting means” for simultaneously adjusting the levels of the output of the high-frequency current and the output of the ultrasound vibration in a state in which the output ratio adjusted by the output ratio adjustment knob 72 is held.

That is, in this embodiment, levels of the output of the ultrasound vibration as an absolute output (reference output) are calibrated around the output adjustment knob 73. Then, by rotating the output adjustment knob 73, the output control device which is interlocked with the rotation shaft of the output adjustment knob 73 is operated, and both the output of the high-frequency current and the output of the ultrasound vibration are adjusted in a state in which the output ratio adjusted by the output ratio adjustment knob 72 is held. In this embodiment, the output of the high-frequency current and the output of the ultrasound vibration are adjusted with the output of the ultrasound vibration as a reference. However, the present invention is not limited to this, and the outputs may be adjusted with the output of the high-frequency current as a reference or the outputs may be adjusted with a sum of the output of the high-frequency current and the output of the ultrasound vibration as a reference.

In addition, in this embodiment, since the output ratio and the magnitude of the output can be adjusted with no steps (continuously) by rotation of each knob, fine-tuning of each output can be easily performed. However, the present invention is not limited to this embodiment, and the surgical operation apparatus may be constructed to adjust each output step by step, or may be constructed to have a display unit such as a liquid crystal panel and adjust the output by inputting a numerical value in the display unit.

In addition, in this embodiment, the output of the high-frequency current and the output of the ultrasound vibration are adjusted by the output ratio adjustment knob 72 and the output adjustment knob 73, as described above. However, it is preferable to provide another adjusting means such as switches that can adjust the output of the high-frequency current and the output of the ultrasound vibration, respectively, in addition to these two adjustment knobs.

Further, if such adjusting means is provided, it is preferable to configure the surgical operation apparatus such that the control device 7 calculates an output ratio based on the value of each output adjusted by the adjusting means and thereby the output ratio adjustment knob 72 and the output adjustment knob 73 are rotated to predetermined positions, respectively, based on the calculated output ratio.

In addition, since the adjustment panel 71 is provided for each of the cutting mode and the coagulation mode, an appropriate output ratio can be set for each mode and treatment can be performed at an appropriate magnitude of output. However, the present invention is not limited to this embodiment, and it is also possible to commonly use one adjustment panel for the cutting mode and the coagulation mode.

In addition, in this embodiment, the output ratio is determined from the output electric power from the high-frequency current generating device and the output electric power from the ultrasound vibration current generating device 4. However, it is also possible to determine an output ratio based on an output current or an output voltage from the high-frequency current generating device 3, an output voltage from the ultrasound vibration current generating device 4, or a vibration amplitude of the ultrasound vibration at the tip of the tip member 22 other than the output electric powers used in this embodiment.

Finally, it is needless to mention that the present invention is not limited to the above-described embodiments but can be modified or improved in various ways within the scope described in claims. For example, it is possible to remove the cutting mode from the surgical operation apparatus of the present invention and construct it only by the coagulation mode and the ultrasound vibration mode. Even such a surgical operation apparatus can be preferably used in the case in which only fragmentation by ultrasound vibration and hemostasis are required such as in case of a surgical operation of liver. According to this modification, a structure of the surgical operation apparatus itself can be simplified.

As described above, according to the present invention, supply of the high-frequency coagulation current and supply of the ultrasound vibration to the tip member can be performed in a cooperatively associated manner. Thus, it is no more necessary to perform complicated manipulations during a surgical operation, and manipulations can be performed conveniently and safely.

Further, when the present invention is configured so that an appropriate coagulation mode ultrasound vibration is supplied during the supply of the high-frequency coagulation current in a cooperatively associated manner, it is possible to preferably prevent adhesion of the burned tissue to the tip member, thus making it possible to use the surgical operation apparatus for a long time.

What is claimed is:

1. A surgical operation apparatus, comprising:
a tip member for outputting ultrasound vibration and/or high-frequency coagulation current;
a handpiece provided with the tip member;
means for supplying at least the high-frequency coagulation current to the tip member;
means for supplying the ultrasound vibration to the tip member, and
control means for controlling the ultrasound vibration supplying means and the high-frequency coagulation current supplying means;
wherein the supply of the high-frequency coagulation current to the tip member and the supply of the ultrasound vibration to the tip member are performed in a cooperatively associated manner as needed.

2. The surgical operation apparatus as claimed in claim 1, wherein the apparatus includes an ultrasound vibration mode for mainly fragmenting tissue in a surgical operation region by outputting the ultrasound vibration from the tip member, and a coagulation mode for mainly performing hemostasis of the tissue by outputting the high-frequency coagulation current from the tip member,
the ultrasound vibration mode and the coagulation mode being switchable by the control means, wherein during the coagulating mode, the ultrasound vibration supplying means supplies a predetermined coagulation mode ultrasound vibration to the tip member.

3. The surgical operation apparatus as claimed in claim 2, wherein the predetermined coagulation mode ultrasound vibration is set to a level that can suppress the tissue from being burnt to be stuck to the tip member.

4. The surgical operation apparatus as claimed in claim 2, wherein the predetermined coagulation mode ultrasound vibration is set to a level that is approximate to 50% of the maximum vibration output of the ultrasound vibration mode.

5. The surgical operation apparatus as claimed in claim 3, a vibration speed of the tip of the tip member is in the range of 220 [cm/s] to 2200 [cm/s] when the coagulation mode ultrasound vibration is supplied to the tip member.

6. The surgical operation apparatus as claimed in claim 1, further comprising means for aspirating liquid or fragmented tissue in the vicinity of the surgical operation region from the tip of the tip member, the aspirating means is adapted to stop during the coagulation mode.

7. The surgical operation apparatus as claimed in claim 1, further comprising means for supplying irrigation liquid to the surgical operation region from the vicinity of the tip of the tip member, and the irrigation liquid supplying means is adapted to supply irrigation liquid in both the ultrasound vibration mode and the coagulation mode.

8. The surgical operation apparatus as claimed in claim 7, wherein the amount of the irrigation liquid to be supplied to the surgical operation region can be set separately in each of the ultrasound vibration mode and the coagulation mode.

9. The surgical operation apparatus as claim in claim 1, the control means includes a hand switch provided on the handpiece to be operated by a hand of an operator.

10. The surgical operation apparatus as claim in claim 1, the control means includes a foot switch to be operated by a foot of an operator.
